(19) United States
(12) Patent Application Publication
(10) Pub. No.: US 2017/0362262 A1
(43) Pub. Date: Dec. 21, 2017

(54) A PROCESS FOR PREPARING HALOGENATED AZAINDOLE COMPOUNDS USING PYBROP

(71) Applicant: ViiV HEALTHCARE UK (NO.4) LIMITED, Brentford (GB)

(72) Inventors: Michael S. BULTMAN, New Brunswick, NJ (US); Benjamin COHEN, New Brunswick, NJ (US); Francisco GONZALEZ-BOBES, New Brunswick, NJ (US); Matthew R. HICKEY, New Brunswick, NJ (US)

(21) Appl. No.: 15/529,572

(22) PCT Filed: Dec. 17, 2015
(86) PCT No.: PCT/US2015/066355

§ 371 Date: May 25, 2017

Related U.S. Application Data
(60) Provisional application No. 62/093,638, filed on Dec. 18, 2014.

(51) Int. Cl. C07F 9/6561; C07D 471/04

(52) U.S. Cl. CPC C07F 9/6561; C07D 471/04

(57) ABSTRACT

A process for preparing halogenated azaindole compounds makes use of a brominating agent PyBroP, together with a dehydrating agent BSA to enhance the selectivity and improve the yield of the final product which is a piperazine prodrug useful as an antiviral.

### CROSS REFERENCE TO RELATED APPLICATION

This application claims the priority of U.S. Provisional Application Ser. No. 62/093,638 filed Dec. 18, 2014 which is herein incorporated by reference in its entirety.

### FIELD OF THE INVENTION

The present invention relates to a process for preparing halogenated azaindole compounds which are used in obtaining HIV attachment inhibitor compounds useful as antivirals. In particular, the invention provides methods of making the piperazine prodrug compound identified as 1-benzoyl-4-[2-[4-methoxy-7-(3-methyl-1H-1,2,4-triazol-1-yl)-1-[(phosphonooxy)methyl]-1H-pyrrolo[2,3-c]pyridin-3-yl]-1,2-dioxoethyl]-piperazine, as well as certain intermediates thereof. The invention also relates to the compounds produced by the processes herein.

### BACKGROUND OF THE INVENTION

HIV-1 (human immunodeficiency virus-1) infection remains a major medical problem, with tens of millions of people still infected worldwide at the end of 2011. The number of cases of HIV and AIDS (Acquired Immuno Deficiency Syndrome) has risen rapidly. In 2005, for example, approximately 5 million new infections were reported and 3.1 million people died from AIDS. Despite continued advances in HIV treatment options, the development of new antiretroviral drugs and regimens continues to represent an important area of unmet medical need due to long-term tolerability concerns and the emergence of viral strains resistant to current therapies. To date, the approved therapies to treat HIV infection fall into 4 general classes: (1) reverse-transcriptase inhibitors, (2) protease inhibitors, (3) integrase inhibitors and (4) entry inhibitors. Examples of available drugs for the treatment of HIV include nucleoside reverse transcriptase (RT) inhibitors or approved single pill combinations: zidovudine (or AZT or RETROVIR®), didanosine (or VIDEX®), stavudine (or ZERIT®), lamivudine (or 3TC or EPIVIR®), zalcitabine (or DDC or HIVID®), abacavir succinate (or ZIAGEN®), Tenofovir disoproxil fumarate salt (or VIREAD®), emtricitabine (or FTC or EMTRIVA®), Combivir® (contains -3TC plus AZT), TRIZIVIR® (contains abacavir, lamivudine, and zidovudine), EPZICOM® (contains abacavir and lamivudine), TRUVADA® (contains VIREAD® and EMTRIVA®); non-nucleoside reverse transcriptase inhibitors: nevirapine (or VIRAMUNE®), delavirdine (or RESCRIPTOR®) and efavirenz (or SUSTIVA®), ATRIPLA® (TRUVADA®+SUSTIVA®), and etravirine, and peptidomimetic protease inhibitors or approved formulations: saquinavir, indinavir, ritonavir, nelfinavir, amprenavir, lopinavir, KALETRA® (lopinavir and Ritonavir), darunavir, atazanavir (REYATAV), and tipranavir (APTIVUS®), and integrase inhibitors such as raltegravir (ISENTRESS®), and entry inhibitors such as enfuvirtide (T-20) (FUZEON®) and maraviroc)(SELZENTRY®).

The identification of potent, orally active antiretrovirals with a unique mechanism of action led to HIV attachment inhibitors, a novel subclass of antiviral compounds, that bind to the HIV surface glycoprotein gp120, and interfere with the interaction between the surface protein gp120 and the host cell receptor CD4. Thus, they prevent HIV from attaching to the human CD4 T-cell, and block HIV replication in the first stage of the HIV life cycle. The properties of HIV attachment inhibitors have been improved in an effort to obtain compounds with maximized utility and efficacy as antiviral agents.

One HIV attachment inhibitor compound, in particular, has now shown considerable prowess against HIV. This compound is identified as 1-(4-benzoyl-piperazin-1-yl)-2-[4-methoxy-7-(3-methyl-[1,2,4]-triazol-1-yl)-1H-pyrralo[2,3-c]pyridine-3-yl]-ethane-1,2-dione, and is set forth and described in U.S. Pat. No. 7,354,924, which is incorporated herein in its entirety. The compound is represented by the formula below:

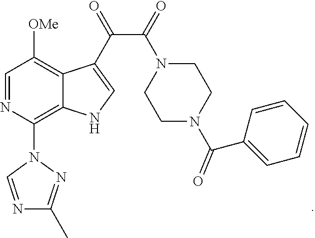 [SMILES: COc1cnc(-n2cnc(C)n2)c2[nH]cc(C(=O)C(=O)N3CCN(C(=O)c4ccccc4)CC3)c12]

The above compound is the parent compound of the prodrug known as 1-benzoyl-4-[2-[4-methoxy-7-(3-methyl-1H-1,2,4-triazol-1-yl)-1-[(phosphonooxy)methyl]-1H-pyrrolo[2,3-c]pyridin-3-yl]-1,2-dioxoethyl]-piperazine. It is set forth and described in U.S. Pat. No. 7,745,625, which is incorporated by reference herein it its entirety. The compound is represented by the formula below:

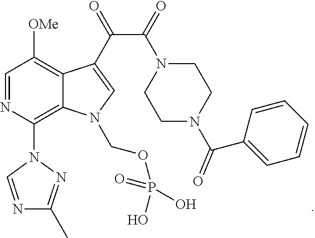 [SMILES: COc1cnc(-n2cnc(C)n2)c2c1c(C(=O)C(=O)N1CCN(C(=O)c3ccccc3)CC1)cn2COP(=O)(O)O]

Various methods for making this prodrug compound have been set forth, including those detailed in the '625 reference. In particular, the '625 reference includes various methods for acylation, alkylation and phosphorylation. Another patent reference, U.S. Pat. No. 8,436,168 entitled “Methods of Making HIV Attachment Inhibitor Prodrug Compound and Intermediates”, also details various procedures for making the piperazine prodrug compound. These include a multi-step process which uses the compound

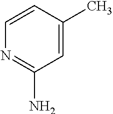

as a starting material, which is subsequently brominated, and then nitrated. Further on, a triazolyl moiety is added to the compound before further attaching the piperazine moiety separated by dual carbonyl groups. Yet another patent reference, U.S. Pat. No. 8,889,869 entitled “Methods of Making HIV Attachment Inhibitor Prodrug Compound and Intermediates”, also details a procedure for making the piperazine prodrug compound. This includes a multi-step process which uses the compound N-sulfonylated pyrrole as a starting material, which is subsequently subjected to a Friedel-Crafts acylation reaction, Pictet-Spengler cyclization, two oxidation reactions followed by bromination, deprotection and a second Friedel-Crafts acylation. Further on, the piperazine moiety is incorporated by amidation of the dual carbonyl groups followed by the copper catalyzed reaction to install the triazolyl moiety.

What is now needed in the art are new methods of making the halogenated azaindole compounds so as to prepare piperazine prodrug compounds which are useful against HIV. The methods should be economical and also be able to produce the halogenated azaindole in high yield and selectivity.

### SUMMARY OF THE INVENTION

In a first embodiment, the invention provides a process for preparing a compound of formula I,

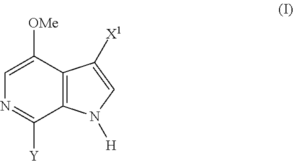

said process comprising the steps of:

(a) performing an oxidation reaction on the compound

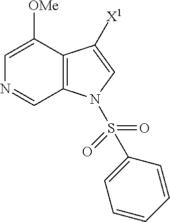

to yield the compound

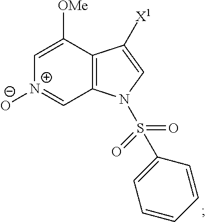

(b) performing a halogenation reaction on the compound obtained in step (a) to obtain the compound

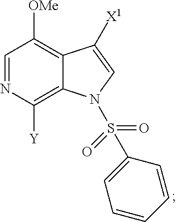

and

(c) performing a deprotection reaction on the compound obtained in step (b) to prepare the compound of formula I above;

wherein X¹ is selected from the group of H,

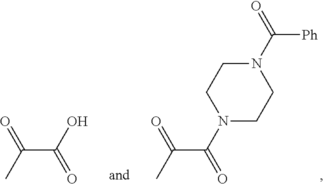

### and Y is Br.

In another embodiment, the invention provides a process for preparing a compound of formula II

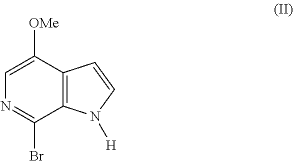 [SMILES: COc1cnc(Br)c2[nH]ccc12]

said process comprising the steps of:

(a) performing an oxidation reaction on the compound

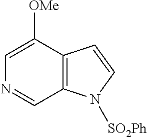

using H₂O₂, phthalic anhydride, and a solvent to yield the compound

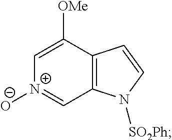

and

(b) performing a bromination reaction on the compound obtained in step (a) using PyBroP and BSA to obtain the compound

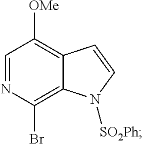

and

(c) performing a deprotection reaction on the compound obtained in step (b) using toluene together with a solvent to prepare the compound of formula II or its salts thereof.

In a further embodiment, the present invention provides a method of making a compound of formula III

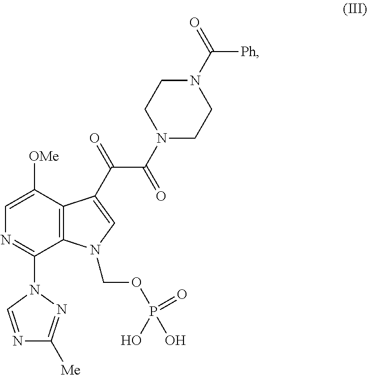 [SMILES: COc1cnc(-n2cnc(C)n2)c2c1c(C(=O)C(=O)N1CCN(C(=O)c3ccccc3)CC1)cn2COP(=O)(O)O]

said process comprising the steps of:

(a) performing an oxidation reaction on compound

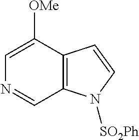

using H₂O₂, phthalic anhydride and dichloromethane to yield the compound

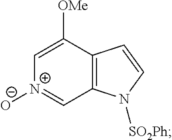

and

(b) performing a bromination reaction on the compound obtained in step (a) using PyBroP and BSA to obtain the compound

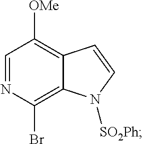

(c) performing a deprotection reaction on the compound obtained in step (b) using toluene together with t-amyl alcohol, followed by crystallization, to obtain the compound

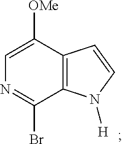 [SMILES: COc1cnc(Br)c2[nH]ccc12]

(d) reacting the compound obtained in step (c) to obtain the compound

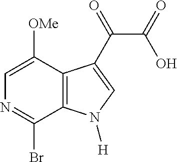 [SMILES: COc1cnc(Br)c2[nH]cc(C(=O)C(=O)O)c12]

followed by reacting it with compound

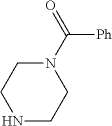 [SMILES: O=C(c1ccccc1)N1CCNCC1]

in an activation reaction to produce compound

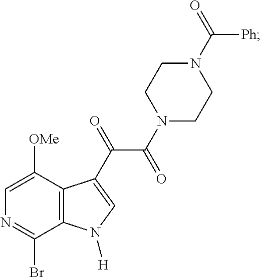 [SMILES: COc1cnc(Br)c2[nH]cc(C(=O)C(=O)N3CCN(C(=O)c4ccccc4)CC3)c12]

and

(e) adding the triazolyl compound

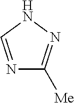

in the presence of Cu ion and a ligand to obtain the compound

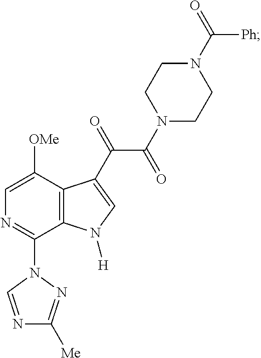 [SMILES: COc1cnc(-n2cnc(C)n2)c2[nH]cc(C(=O)C(=O)N3CCN(C(=O)c4ccccc4)CC3)c12]

wherein said ligand is selected from the group of 1,2-diaminocyclohexane, trans-1,2-diaminocyclohexane, cis-/trans-diaminocyclohexane, cis-N,N′-dimethyl-1,2-diaminocyclohexane, trans-N,N′-dimethyl-1,2-diaminocyclohexane, cis-/trans-N,N′-dimethyl-1,2-diaminocyclohexane, 1,2-diaminoethane, N,N′-dimethyl-1,2-diaminoethane, 1,10-phenanthroline, 4,7-diphenyl-1,10-phenantroline, 5-methyl-1,10-phenanthroline, 5-chloro-1,10-phenantroline, and 5-nitro-1,10-phenanthroline; and

(f) reacting the compound obtained in step (e) with (tert-BuO)₂POOCH₂Cl to produce the compound

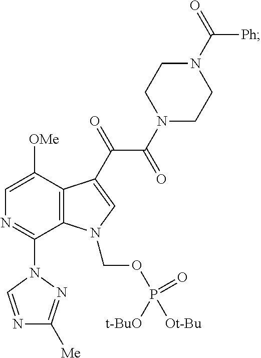 [SMILES: COc1cnc(-n2cnc(C)n2)c2c1c(C(=O)C(=O)N1CCN(C(=O)c3ccccc3)CC1)cn2COP(=O)(OC(C)(C)C)OC(C)(C)C]

and reacting the compound obtained in step (f) with an acid, for example acetic acid, to yield the compound of formula III above.

The invention in further embodiments is also directed to each of the compounds of formulas I, II and III herein which are produced by the processes herein set forth.

The present invention is directed to these, as well as other important ends, hereinafter described.

### DETAILED DESCRIPTION OF THE EMBODIMENTS

It will be understood that any given exemplary embodiment can be combined with one or more additional exemplary embodiments. As used herein, the singular forms “a”, “an”, and “the” include plural reference unless the context clearly dictates otherwise.

Unless otherwise specifically set forth, many reagents have been identified herein by their commonly accepted letter abbreviations in the art for ease of reference.

In addition, unless otherwise specifically set forth elsewhere in the application, the following terms may be used herein, and shall have the following meanings:

An “alkyl” group refers to a saturated aliphatic hydrocarbon including straight chain and branched chain groups. Preferably, the alkyl group has 1 to 20 carbon atoms (whenever a numerical range; e.g., “1-20”, is stated herein, it means that the group, in this case the alkyl group may contain 1 carbon atom, 2 carbon atoms, 3 carbon atoms, etc. up to and including 20 carbon atoms). More preferably, it is a medium size alkyl having 1 to 10 carbon atoms. Most preferably, it is a lower alkyl having 1 to 4 carbon atoms. The alkyl group may be substituted or unsubstituted.

The term “C₁₋₆alkyl” as used herein and in the claims means straight or branched chain alkyl groups with up to and including 6 carbon atoms, such as methyl, ethyl, propyl, isopropyl, butyl, isobutyl, t-butyl, amyl, hexyl and the like.

An “aryl” “Aryl” or “Ar” group refers to an all carbon monocyclic or fused-ring polycyclic (i.e., rings which share adjacent pairs of carbon atoms) groups having a completely conjugated pi-electron system. Examples, without limitation, of aryl groups are phenyl, napthalenyl and anthracenyl. The aryl group may be substituted or unsubstituted.

The abbreviations used in the present application are well-known to those skilled in the art. Some of the abbreviations used are as follows:

PyBroP—Bromo-tris-pyrrolidino phosphoniumhexafluorophosphate

DIPEA or Hünig's base=Diisopropylethylamine

K₃PO₄=potassium phosphate tribasic

Ph=Phenyl

H₂O₂: Hydrogen peroxide

BSA: N,O-Bis(trimethylsilyl)acetamide

t-amyl alcohol: 2-methyl-2-butanol

t-Bu: tert-butyl

Tris: 2-amino-2-(hydroxymethyl)propane-1,3-diol

In a first aspect, the present invention provides a process for preparing a compound of formula I,

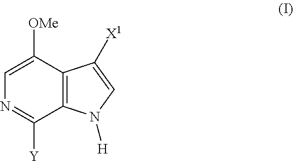

said process comprising the steps of:

(a) performing an oxidation reaction on the compound

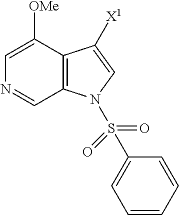

to yield the compound

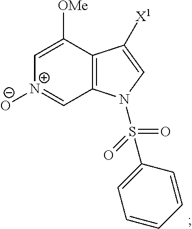

(b) performing a halogenation reaction on the compound obtained in step (a) to obtain the compound

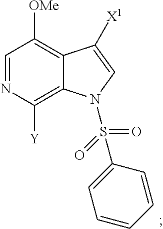

and

(c) performing a deprotection reaction on the compound obtained in step (b) to prepare the compound of formula I above;

wherein X¹ is selected from the group of H,

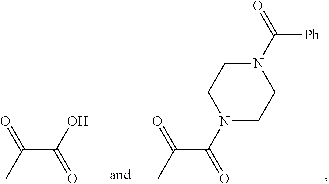

### and Y is Br.

In a first embodiment of the first aspect, the oxidation reaction is carried out using oxidizing agents selected from the group of catalytic methyltrioxorhenium (MTO) and hydrogen peroxide urea complex (UHP), m-CPBA, a mixture of Ac₂O and H₂O₂, and a mixture of phthalic anhydride and H₂O₂.

In a second embodiment of the first aspect the compound

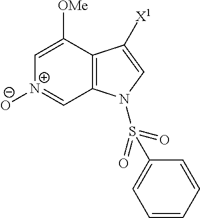

obtained in step (a) of the first aspect is treated with aqueous Na₂SO₃ followed by addition of aqueous K₃PO₄.

In a third embodiment of the first aspect, the compound

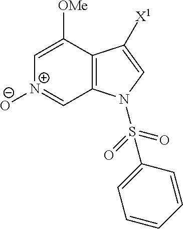

obtained in step (a) of the first aspect is a crystalline solid with about 85% yield and >about 99 area % purity.

In a fourth embodiment of the first aspect, the halogenation reaction is a bromination reaction carried out using PyBroP and a solvent selected from the group of toluene, trifluorotoluene, dichloromethane, chloroform, tetrahydrofuran, and acetonitrile. The reaction may also optionally be carried out with PyBroP and a base and solvent combination selected from the group of K₃PO₄ and Ph-CF₃, N,N,-4-trimethylaniline and Ph-CF₃, and DIPEA (N,N-diisopropylethylamine), and toluene. Other bases may be selected from the group consisting of organic and inorganic bases, including metal carbonates, phosphates, and tertiary alkylamines.

In a fifth embodiment of the first aspect, the halogenation reaction is a bromination reaction carried out in the presence of a dehydrating agent such as BSA or molecular sieves. It is highly preferred to utilize BSA in the halogenation step, along with the PyBrop. Unlike earlier disclosures of the use of a strong base such as NaOH and/or K₃PO₄ with the PyBrop, BSA is not a base and ultimately provided an unexpected advantage overall. The BSA, while functioning essentially as a dehydrating agent, also enhanced selectivity, and provided for optimal conversion and yield. Without being bound by any particular theory, it appears that the BSA prevented reaction stalling via unproductive consumption of the PyBroP.

In a sixth embodiment of the first aspect, the deprotection reaction is carried out using toluene together with t-amyl alcohol.

In a seventh embodiment of the first aspect, the compound of formula I

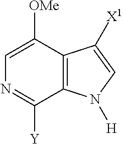

is obtained with a yield ranging from about 62% to 69% and purity of > about 99%.

In a second aspect the present invention provides a process for preparing a compound of formula II

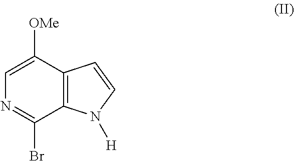 [SMILES: COc1cnc(Br)c2[nH]ccc12]

said process comprising the steps of:

(a) performing an oxidation reaction on the compound

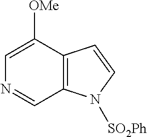

using H₂O₂, phthalic anhydride, and solvent to yield the compound

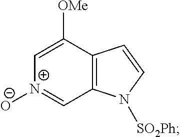

and

(b) performing a bromination reaction on the compound obtained in step (a) using PyBroP and BSA to obtain the compound

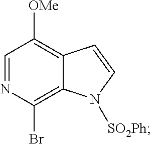

and

(c) performing a deprotection reaction on the compound obtained in step (b) using toluene together with a solvent, followed by crystallization, to prepare the compound of formula II or its salts thereof.

In a third aspect the present invention provides a method of making a compound of formula III

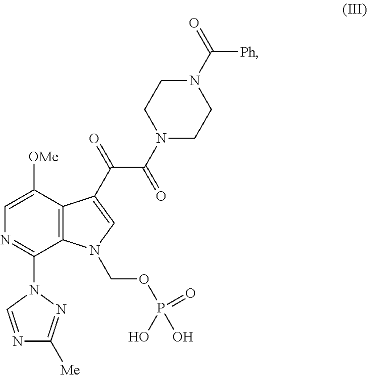 [SMILES: COc1cnc(-n2cnc(C)n2)c2c1c(C(=O)C(=O)N1CCN(C(=O)c3ccccc3)CC1)cn2COP(=O)(O)O]

said process comprising the steps of:

(a) performing an oxidation reaction on the compound

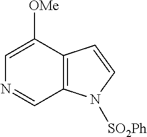

using H₂O₂, phthalic anhydride and dichloromethane to yield the compound

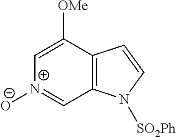

; and

(b) performing a bromination reaction on the compound obtained in step (a) using PyBroP and BSA to obtain the compound

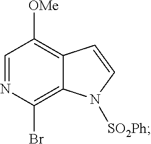

(c) performing a deprotection reaction on the compound obtained in step (b) using toluene together with t-amyl alcohol, followed by crystallization, to obtain the compound

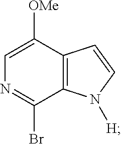

(d) reacting the compound obtained in step (c) to obtain the compound

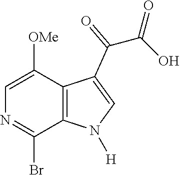 [SMILES: COc1cnc(Br)c2[nH]cc(C(=O)C(=O)O)c12]

followed by reacting it with compound

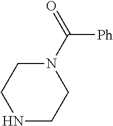 [SMILES: O=C(c1ccccc1)N1CCNCC1]

in an activation reaction to produce the compound

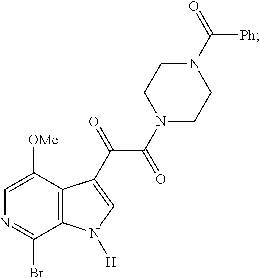 [SMILES: COc1cnc(Br)c2[nH]cc(C(=O)C(=O)N3CCN(C(=O)c4ccccc4)CC3)c12]

and

(e) adding the triazolyl compound

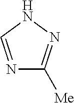

in the presence of Cu ion and a ligand to obtain the compound

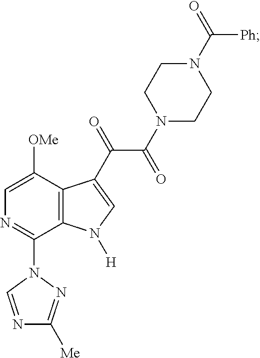 [SMILES: COc1cnc(-n2cnc(C)n2)c2[nH]cc(C(=O)C(=O)N3CCN(C(=O)c4ccccc4)CC3)c12]

wherein said ligand is selected from the group of 1,2-diaminocyclohexane, trans-1,2-diaminocyclohexane, cis-/trans-diaminocyclohexane, cis-N,N′-dimethyl-1,2-diaminocyclohexane, trans-N,N′-dimethyl-1,2-diaminocyclohexane, cis-/trans-N,N′-dimethyl-1,2-diaminocyclohexane, 1,2-diaminoethane, N,N′-dimethyl-1,2-diaminoethane, 1,10-phenanthroline, 4,7-diphenyl-1,10-phenantroline, 5-methyl-1,10-phenanthroline, 5-chloro-1,10-phenantroline, and 5-nitro-1,10-phenanthroline; and

(f) reacting the compound obtained in step (e) with (tert-BuO)₂POOCH₂Cl to produce the compound

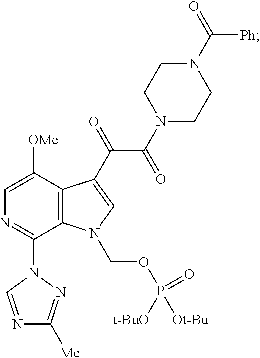 [SMILES: COc1cnc(-n2cnc(C)n2)c2c1c(C(=O)C(=O)N1CCN(C(=O)c3ccccc3)CC1)cn2COP(=O)(OC(C)(C)C)OC(C)(C)C]

and reacting the compound obtained in step (f) with an acid, such as acetic acid, to yield the compound of formula III above.

### EXAMPLES

The present invention will now be described in connection with certain embodiments which are not intended to limit its scope. On the contrary, the present invention covers all alternatives, modifications, and equivalents as can be included within the scope of the claims. Thus, the following examples, which include specific embodiments, will illustrate one practice of the present invention, it being understood that the examples are for the purposes of illustration of certain embodiments and are presented to provide what is believed to be the most useful and readily understood description of its procedures and conceptual aspects.

The compounds of the present invention may be prepared using the reactions and techniques described in this section, as well as other synthetic methods which may be available to those of ordinary skill in the art. The reactions are performed in solvents appropriate to the reagents and materials employed and suitable for the transformation being affected. Also, in the description of the synthetic methods described below, it is to be understood that all proposed reaction conditions, including choice of solvents, reaction temperature, duration of the experiment and workup procedures, are chosen to be the conditions standard for that reaction, which should be readily recognized by one skilled in the art. It is understood by one skilled in the art of organic synthesis that the functionality present on various portions of the molecule must be compatible with the reagents and reactions proposed. Such restrictions to the substituents which are compatible with the reaction conditions will be readily apparent to one skilled in the art and alternate methods must then be used.

In a preferred embodiment of the invention, the synthesis of the halogenated azaindole compounds can be set forth in the following schematic representation - Scheme I.

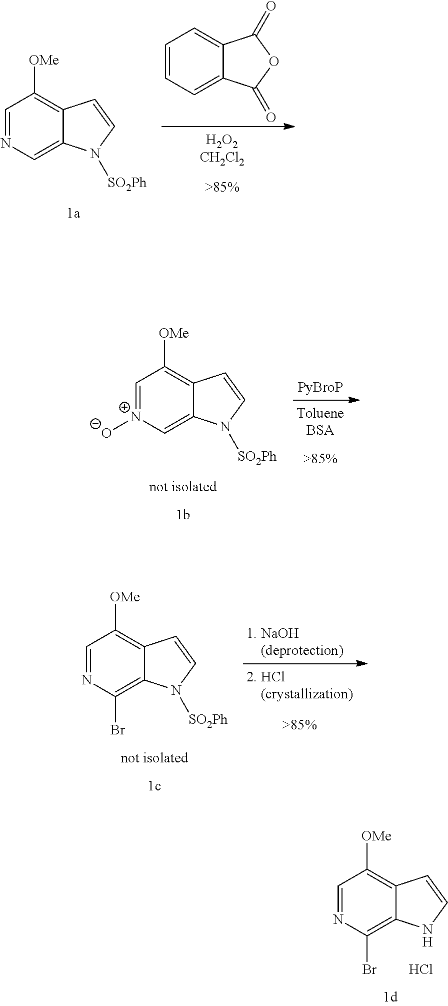

All reagents were used as received without further purification. Reaction progress and final product purity was monitored using HPLC conditions, Table 1, using an Ascentis Express C18, 2.7 μm 4.6×150 mm column at 25° C. Mobile Phase A: 0.01M NH₄OAc in H₂O:MeOH (80:20), Mobile phase B: 0.01 NH₄OAc in H₂O:MeCN:MeOH (5:75:20), 1.0 mL/min. Gradient:

TABLE 1
HPLC Conditions

| | | |
|---|---|---|
| | Mobile Phase | |
| Time | Composition | Gradient |

| (minutes) | % A | % B | Profile |
|---|---|---|---|
| 0.0 | 100.0 | 0.0 | Initial |
| 5.0 | 70.0 | 30.0 | Linear |
| 20.0 | 55.0 | 45.0 | Linear |
| 25.0 | 0.0 | 100.0 | Linear |
| 30.0 | 0.0 | 100.0 | Hold |

7-Bromo-4-methoxy-1H-pyrrolo[2,3-c]pyridine hydrochloride monohydrate (Compound 1d). CH₂Cl₂ (3724 kg), Compound 1a (200 kg, 1.0 equiv) and phthalic anhydride (134 kg, 1.3 equiv) were charged to an 8000 L glass lined vessel. The resulting mixture was heated to 35° C. A 35% w/w aqueous solution of hydrogen peroxide (80.9 kg, 1.2 equiv) was added via pump over 2 hours. The resulting suspension was stirred at 35-37° C. for an additional 2 hours, then sampled and analyzed by HPLC to determine the reaction progress. Once the oxidation reaction was deemed complete, the mixture was cooled to 10° C. The reaction was quenched by controlled addition of a solution of sodium sulfite (88 kg) in water (1400 kg) such that the internal temperature remained below 20° C. The resulting biphasic mixture was stirred vigorously at 20° C. for 2 hours to ensure complete reduction of any residual oxidant. A solution of K₃PO₄ (380 kg) in water (1400 kg) was then added to the quenched reaction mixture and the biphasic mixture stirred at 20° C. for 2 hours. The top aqueous phase was discarded and the product rich organic phase was washed with water (1400 kg). The bottom product rich organic phase was transferred to a clean 8000 L reactor.

Toluene (1740 kg) was added, and the batch concentrated at <0.075 MPa while maintaining the jacket temperature below 40° C. to a final volume of 3000 L. Toluene (1740 kg) was added and the batch concentrated to a final batch volume of 3000 L. N,O-Bis(trimethylsilyl)acetamide (142 kg, 1.0 equiv) was added and the batch cooled to 10° C. PyBroP (390 kg, 1.2 equiv) was added to the batch in a single portion and the resulting mixture was stirred for 15 hours, then sampled and analyzed. During this time the reaction mixture changed from a thin solid suspension to a biphasic mixture composed of a heavy oil phase (bottom) and a clear colorless liquid phase (top).

After completion of the bromination reaction, 2-methyl-2-butanol (1620 kg) was added and the mixture was concentrated to 3000 L. A second portion of 2-methyl-2-butanol (1620 kg) was added and distillation to 3000 L was repeated. A solution of sodium hydroxide (200 kg) in water (1000 kg) was added to the reactor at such a rate that the internal temperature was maintained below 40° C. The resulting mixture was then transferred to an 8000 L stainless steel vessel and heated to 75° C. for 10 hours. The reaction mixture was cooled to 20° C., the phases were allowed to split and were then separated. The aqueous layer was discarded. The top phase (product-rich) was washed sequentially with water (1000 L), a solution of K₂HPO₄ (100 kg) in water (1000 L), and water (1000 L).

The organic stream was transferred to an 8000 L glass lined vessel through a polish filter (1 μm), then concentrated (T≦40° C., <0.1 MPa) to a final volume of 2000 L. 2-Methyl-2-butanol (1620 kg) was added and the resulting solution was again concentrated under vacuum to 2000 L. The resulting mixture was heated to 35° C., and then aqueous HCl (86 kg, 35 w/w %, 1.2 equiv) was added over 2 hours. The resulting suspension was cooled to 20° C. over 1 h, then stirred for 2 hours. The product was collected by centrifugation, washed twice with toluene (436 kg each) and dried at 50° C. at <0.1 MPa to afford the brominated azaindole ld as an off-white solid, 124.8 kg (62.6% corrected yield).
m.p.: 160° C. (decomposition)
¹H NMR (500 MHz, DMSO-d6) δ: 12.80 (s, 1 H), 7.84 (s, br, 1 H), 7.68 (s, 1 H), 6.99 (s, br, 4 H), 6.73 (s, br, 1 H), 3.97 (s, 3 H). ¹³C NMR (125 MHz, DMSO-d6) δ: 149.8, 133.7, 131.8, 126.8, 115.8, 114.0, 101.0, 56.8. HRMS [M+H; ESI-ORBITRAP] calc. for C₈H₈BrN₂O (as free base): 226.9820; found: 226.9813.

Thus, the halogenated azaindole compounds and the reactions described above can be used in the production of the piperazine prodrug compound as shown in Scheme II below. Also, in Scheme II, particularly 1e may be converted to 1i using the schemes described in PCT application number PCT/US2013/024880 filed Feb. 6, 2013, entitled “Methods for the Preparation of HIV Attachment Inhibitor Piperazine Prodrug Compound”, and incorporated herein in its entirety.

A Friedel-Crafts acylation followed by hydrolysis and amidation produced intermediate 1f. The triazole substituent is then incorporated via a copper-catalyzed Ullmann-Goldberg-Buchwald cross-coupling reaction leading to the formation of 1g.

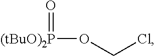

Attachment of the phosphate moiety using followed by hydrolysis and crystallization afford the drug substance 1i.

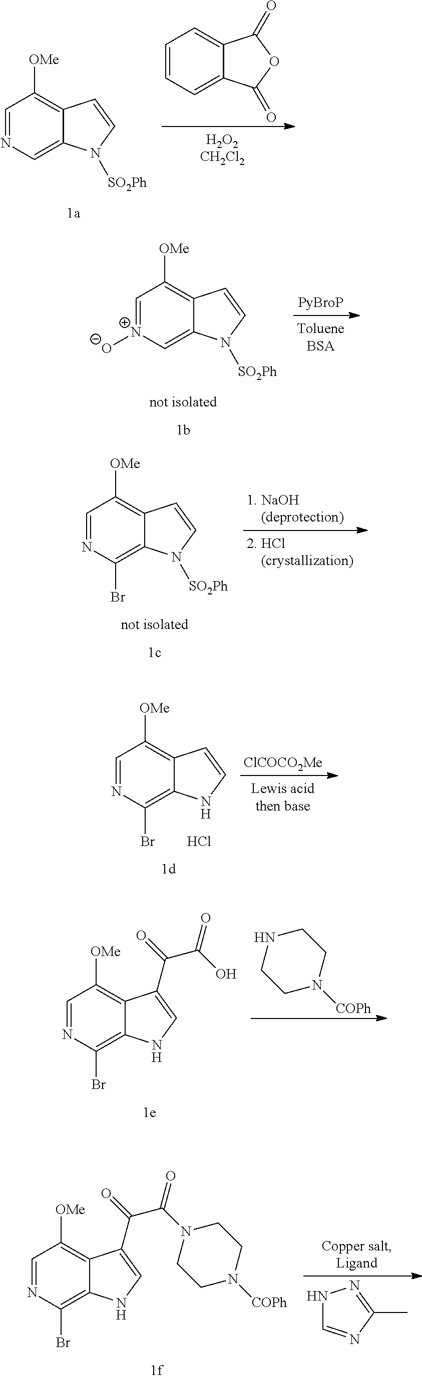

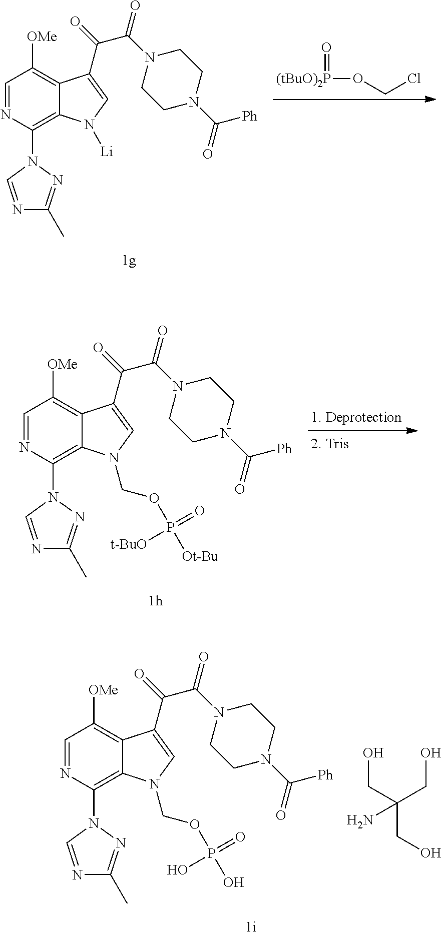

This approach presents the following advantages which are important for the performance of the chemistry: (a) improved safety by avoiding isolation of a highly energetic and mutagenic N-oxide; (b) reduced cost by using phthalic anhydride and aqueous H₂O₂ in the preparation of the N-oxide; (c) improved yield and reduced material balance variability during the oxidation by implementing a slow H₂O₂ addition and modifying the work-up; (d) addressed reaction stalling in the bromination reaction and demonstrated that BSA can be used as an additive for optimal conversion, selectivity, and yield; and (e) eliminated the GTI (genotoxic impurity) concern related to isopropylsulfonate, the need for iterative back extractions, and the slow filtration of the hydrochloride salt. In addition, the use of both PyBroP and BSA to effect bromination of heterocyclic N-oxides represents an important finding which may be applicable to piperazine prodrug compounds which are generally high dose therapeutic agents, and the processes set forth herein reduce the overall cost to manufacture and can provide increased access to such types of related substrates.

It will be evident to one skilled in the art that the present invention is not limited to the foregoing disclosure, and that it can be embodied in other specific forms without departing from the essential attributes thereof. It is therefore desired that the instant disclosure be considered in all respects as illustrative and not restrictive, reference being made to the appended claims, rather than to the foregoing disclosure, and all changes which come within the meaning and range of equivalency of the claims are therefore intended to be embraced therein.

What is claimed is:

1. A process for preparing a compound of formula I, 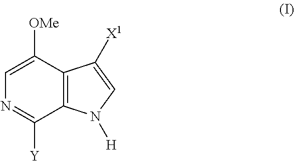
said process comprising the steps of:
(a) performing an oxidation reaction on the compound 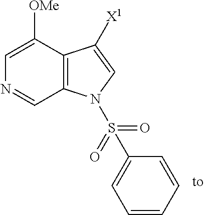
to yield the compound 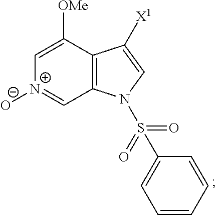
(b) performing a halogenation reaction on the compound obtained in step (a) to obtain the compound 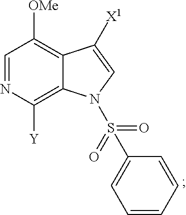
and
(c) performing a deprotection reaction on the compound obtained in step (b) to prepare the compound of formula I above;
wherein X¹ is selected from the group of H, 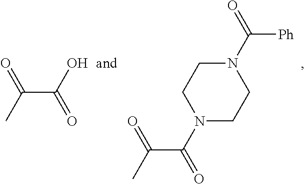
and Y is Br.

2. The process of claim 1, wherein said oxidation reaction is carried out using oxidizing agents selected from the group of catalytic methyltrioxorhenium (MTO) and hydrogen peroxide urea complex (UHP), m-CPBA, a mixture of Ac₂O and H₂O₂, and a mixture of phthalic anhydride and H₂O₂.

3. The process of claim 1, wherein the compound 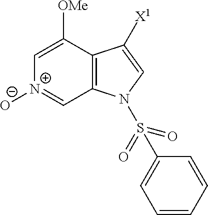
obtained in step (a) is treated with aqueous Na₂SO₃ followed by addition of aqueous K₃PO₄.

4. The process of claim 1, wherein the compound 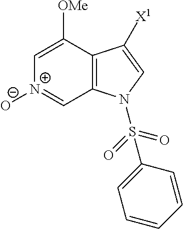
obtained in step (a) is a crystalline solid with about 85% yield and >about 99% purity.

5. The process of claim 1, wherein said halogenation reaction is a bromination reaction carried out using PyBroP and a solvent selected from the group of toluene, trifluorotoluene, dichloromethane, chloroform, tetrahydrofuran, and acetonitrile.

6. The process of claim 1, wherein said halogenation reaction is carried out using PyBroP in the presence of a dehydrating agent which is selected from the group of BSA and molecular sieves.

7. The process of claim 1, wherein said deprotection reaction is carried out using toluene together with t-amyl alcohol.

8. The process of claim 1, wherein the compound of formula I 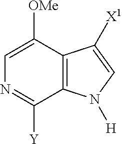
is obtained with a yield ranging from about 62% to 69% and purity of >about 99 area %.

9. A process for preparing a compound of formula II 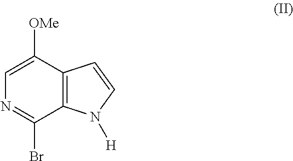 [SMILES: COc1cnc(Br)c2[nH]ccc12]
said process comprising the steps of:
(a) performing an oxidation reaction on the compound 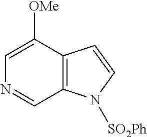
using H₂O₂, phthalic anhydride and solvent to yield the compound 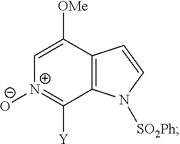
and
(b) performing a bromination reaction on the compound obtained in step (a) using PyBroP and BSA to obtain the compound 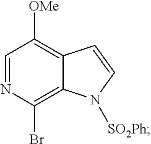
and
(c) performing a deprotection reaction on the compound obtained in step (b) using toluene together with solvent to prepare the compound of formula II or its salts thereof.

10. A method of making a compound of formula III 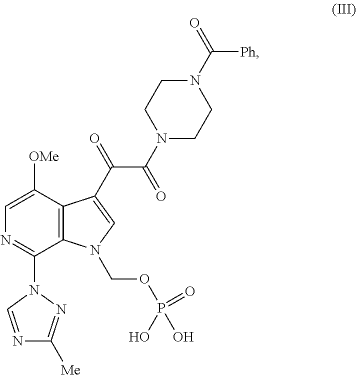 [SMILES: COc1cnc(-n2cnc(C)n2)c2c1c(C(=O)C(=O)N1CCN(C(=O)c3ccccc3)CC1)cn2COP(=O)(O)O]
said process comprising the steps of:
(a) performing an oxidation reaction on the compound 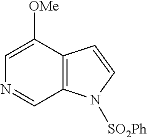
using H₂O₂, phthalic anhydride and dichloromethane to yield the compound 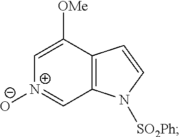
and
(b) performing a bromination reaction on the compound obtained in step (a) using PyBroP and BSA to obtain the compound 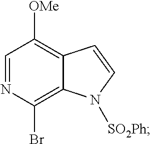
(c) performing a deprotection reaction on the compound obtained in step (b) using toluene in conjunction with t-amyl alcohol, followed by crystallization, to obtain the compound 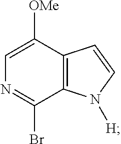
(d) reacting the compound obtained in step (c) to obtain the compound 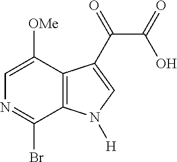 [SMILES: COc1cnc(Br)c2[nH]cc(C(=O)C(=O)O)c12]
followed by reacting it with compound 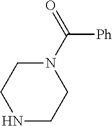 [SMILES: O=C(c1ccccc1)N1CCNCC1]
in an activation reaction to produce the compound 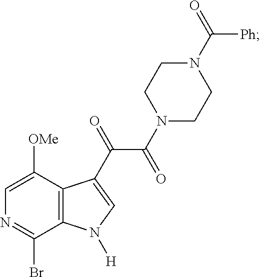 [SMILES: COc1cnc(Br)c2[nH]cc(C(=O)C(=O)N3CCN(C(=O)c4ccccc4)CC3)c12]
and
(e) adding the triazolyl compound 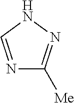
in the presence of Cu ion and a ligand to obtain the compound 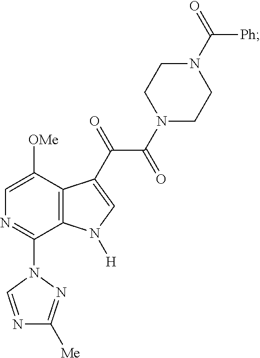 [SMILES: COc1cnc(-n2cnc(C)n2)c2[nH]cc(C(=O)C(=O)N3CCN(C(=O)c4ccccc4)CC3)c12]
wherein said ligand is selected from the group of 1,2-diaminocyclohexane, trans-1,2-diaminocyclohexane, cis-/trans-diaminocyclohexane, cis-N,N′-dimethyl-1,2-diaminocyclohexane, trans-N,N′-dimethyl-1,2-diaminocyclohexane, cis-/trans-N,N′-dimethyl-1,2-diaminocyclohexane, 1,2-diaminoethane, N,N′-dimethyl-1,2-diaminoethane, 1,10-phenanthroline, 4,7-diphenyl-1,10-phenantroline, 5-methyl-1,10-phenanthroline, 5-chloro-1,10-phenantroline, and 5-nitro-1,10-phenanthroline; and
(f) reacting the compound obtained in step (e) with (tert-BuO)₂POOCH₂Cl to produce the compound 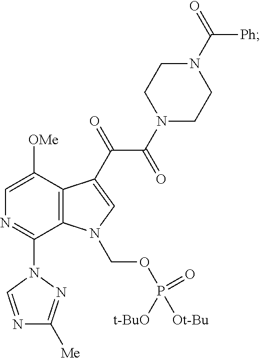 [SMILES: COc1cnc(-n2cnc(C)n2)c2c1c(C(=O)C(=O)N1CCN(C(=O)c3ccccc3)CC1)cn2COP(=O)(OC(C)(C)C)OC(C)(C)C]
and reacting the compound obtained in step f with an acid to yield compound of formula III above.

11. The process of claim 6, wherein said halogenation reaction is carried out in the presence of BSA.
